(19) United States
(12) Patent Application Publication
(10) Pub. No.: US 2024/0009446 A1
(43) Pub. Date: Jan. 11, 2024

(54) HEAT EXCHANGE APPARATUS AND METHOD OF OPERATION

(71) Applicant: CardioQuip, LLC, College Station, TX (US)

(72) Inventors: William Stewart, College Station, TX (US); John Dean, College Station, TX (US); Douglas Platt, College Station, TX (US)

(73) Assignee: CardioQuip, LLC, College Station, TX (US)

(21) Appl. No.: 18/346,184

(22) Filed: Jun. 30, 2023

Related U.S. Application Data
(60) Provisional application No. 63/358,626, filed on Jul. 6, 2022.

(51) Int. Cl. A61M 60/849; A61M 60/90

(52) U.S. Cl. CPC A61M 60/849; A61M 60/90; A61M 2205/3368; A61M 2205/36

(57) ABSTRACT

A heat exchange system for controlling the temperature of the blood of a patient. The heat exchange system comprises a disposable component having a blood inlet to receive blood from the patient and a blood outlet to return blood to the patient, and a permanent component. The permanent component includes a first housing having an inlet and an outlet to exchange thermal fluid with an external device and a second housing having an inlet and an outlet to exchange thermal fluid with the external device. The disposable component is inserted between the first housing and the second housing, such that in a closed position of the permanent component, a first surface of the disposable component is pressed into contact with a surface of the first housing and a second surface of the disposable component is pressed into contact with a surface of the second housing.

### CROSS-REFERENCE TO RELATED APPLICATION(S) AND CLAIM OF PRIORITY

The present application is related to U.S. Provisional Patent No. 63/358,626, filed 6 Jul. 2022, entitled “HEAT EXCHANGER AND METHOD OF OPERATION”. Provisional Patent No. 63/358,626 is assigned to the assignee of the present application and is hereby incorporated by reference into the present application as if fully set forth herein. The present application hereby claims priority under 35 U.S.C. § 119(e) to U.S. Provisional Patent No. 63/358,626.

### TECHNICAL FIELD

The present application relates generally to a heat exchanger used with patient thermal management devices.

### BACKGROUND

Patient temperature management devices are used in a variety of medical applications, including emergency medical services, extracorporeal membrane oxygenation, intensive care unit (ICU) treatment, cardiovascular perfusion, and targeted temperature management, among others. Following a cardiac arrest, for instance, a patient may be cooled to improve neurological outcomes. These temperature management devices take many forms, including: i) standalone heat exchangers that use metal channels as the thermal interface between a thermal fluid and blood; ii) oxygenator-heat exchanger combination products that permanently integrate a plastic or metallic heat exchanger to a reservoir and oxygenator, iii) catheter products that flow temperature-controlled fluid around an injection-site catheter, and the like. These designs share a common feature: two isolated chambers, one for blood and one for temperature-controlled fluid, separated by a thermally conductive interface.

These devices have varying levels of efficacy, depending on the clinical need presented. Oxygenator-heat-exchanger combinations offer the convenience of being a single solution for perfusion needs and can reduce tubing management associated with separate products. Standalone heat exchangers are often used if an oxygenator does not include a heat exchanger. Standalone products may also be used if combination devices are incompatible with other devices or patients require greater thermal transfer from a larger heat exchanger. Catheter products offer great performance in a very specific use case and can provide thermal management without perfusing the patient.

Although the previously mentioned products provide benefits, they share numerous flaws, including cost, associated waste, and complexity. These devices are single-use products, and, in the case of the oxygenator combination product, the heat exchanger may be the most expensive portion of the product. These devices add significant cost and waste to each procedure and require extra tubing and effort to set up the device with a heater-cooler. Additionally, the thermal interface may present risks to the patient in the form of leaks that compromise the sterile condition of the system and expose the patient to disinfecting chemicals. Lastly, the disposal process typically results in the loss of small volumes of temperature-controlled fluid inside the heat exchanger, requiring user intervention to refill the heater-cooler.

Most temperature-control devices in the market are water-based systems, which are under scrutiny by FDA as they may harbor bacteria in their internal water paths. These bacteria can then be aerosolized into the operating room and may cause patient infection. FDA has asked manufacturers to revalidate cleaning and disinfection procedures to ensure that the water quality of water-based systems never exceeds a specific bacterial contamination threshold. Consequently, patient temperature management devices will either require constant levels of disinfecting chemicals in their devices or move away from water as the temperature-controlled fluid. Most heat exchangers are only compatible with water, and added disinfecting chemicals or other fluids may breach the thermal interface with the patient's blood. Heat exchangers that are compatible with disinfectants require strict control of chemical concentrations to prevent leaks. Although the heat exchangers can be evaluated for compatibility with new fluids, there is broad market concern regarding only having a single barrier between the temperature-controlled fluid and the blood of the patient.

Due to associated excessive costs, waste, complexity, and concerns with fluid compatibility, there is a need for a new heat exchanger design that can provide an affordable, efficacious, and safe solution for the industry.

### SUMMARY

To address the above-discussed deficiencies of the prior art, it is a primary object of the present disclosure to provide a heat exchange system for controlling the temperature of a patient's blood. In an embodiment, the heat exchange system comprises a disposable component having a blood inlet configured to receive blood from the patient and a blood outlet configured to return blood to the patient, and a permanent component. The permanent component includes: a first housing having an inlet and an outlet configured to exchange thermal fluid with an external device; and a second housing having an inlet and an outlet configured to exchange thermal fluid with the external device. The disposable component is configured to be inserted between the first housing and the second housing, such that in a closed position of the permanent component, a first surface of the disposable component is pressed into contact with a surface of the first housing and a second surface of the disposable component is pressed into contact with a surface of the second housing.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a block diagram illustrating a heat exchange system according to an embodiment of the present disclosure.

FIG. 2 is a perspective view of a side of a permanent component of a heat exchange system according to an embodiment of the present disclosure.

FIG. 3 is a perspective view of the rear of a permanent component of a heat exchange system according to an embodiment of the present disclosure.

FIG. 4 is a perspective view of a disposable component of a heat exchange system according to an embodiment of the present disclosure.

FIG. 5 is a cutaway view of a disposable component of a heat exchange system according to an embodiment of the present disclosure.

FIG. 6 is a block diagram illustrating a heat exchange system according to an alternate embodiment of the present disclosure.

FIG. 7 is a block diagram illustrating a heat exchange system according to an alternate embodiment of the present disclosure.

### DETAILED DESCRIPTION

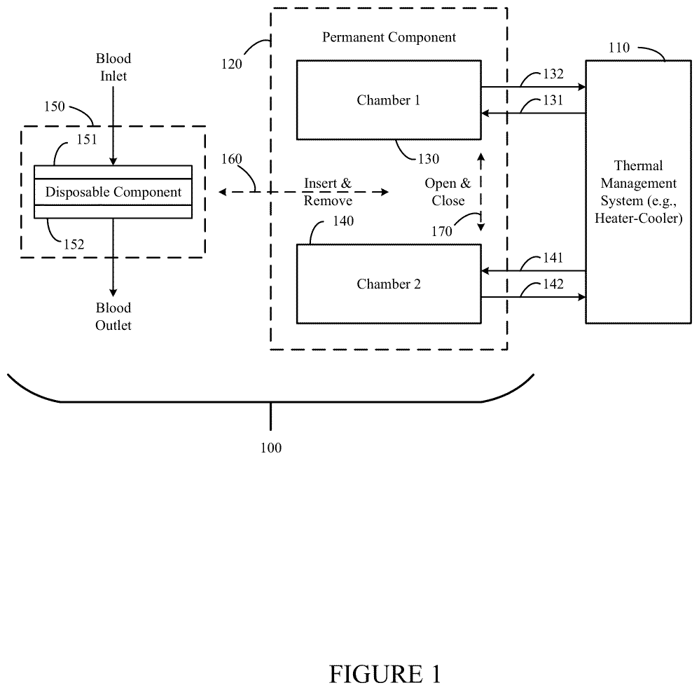

FIGS. 1 through 7, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged heat exchanger.

The present disclosure describes a heat exchange system that provides patient temperature management in a variety of medical applications, such as emergency medical services (EMS), extracorporeal membrane oxygenation (ECMO), and intensive care unit (ICU) treatment, among others. The disclosed system achieves temperature regulation using a disposable component and a permanent (or non-disposable) component. The two-part heat exchanger provides thermal transfer in perfusion applications. The two-part heat exchanger is used with patient thermal management devices (e.g., heater-cooler devices) intended to control patient or blood temperature through a blood-fluid interface.

In an example embodiment, the permanent component of the two-part heat exchanger may be coupled to a patient thermal management device. The permanent component may comprise two flexibly coupled housings that may clamp around the disposable component, which may be a disposable blood-contacting device. The permanent component may require external cleanings after each clinical use and internal cleanings at a manufacturer-specified interval. The permanent component may circulate temperature-controlled fluid through one or more separate chambers to recirculate the fluid back to the heater-cooler device. The permanent component may be rated for water, chemical additives (e.g., hydrogen peroxide, chloramine, free chlorine, etc.), and other temperature-controlled fluids, such as glycol-based solutions. Advantageously, a significant benefit provided by the proposed design is the double-layered isolation of any chemicals used for fluid temperature maintenance from the rest of the perfusion system.

In an example embodiment, the disposable component may comprise a single-use component distributed in a perfusion kit purchased by the user. The disposable component may comprise a chamber with a high surface area of flowing blood for better thermal transfer. According to the principles of the present disclosure, on one side or both sides of the disposable component, there may be a thermally conductive membrane that adheres only to the disposable component that aids in thermal transfer when placed in contact with the permanent component. At the start of use, the operator may clamp or lock the disposable component onto the housings of the permanent component. This enables thermal transfer between the disposable component and the permanent component. At the end of a perfusion operation, the disposable component may be removed and discarded, thus retaining the permanent component and thermal-transfer fluid for future use.

In an example embodiment, the permanent component and the disposable component may comprise a thermally conductive metal (e.g., stainless steel) to provide adequate thermal transfer. In some embodiments, the permanent component may comprise multiple housings, each of which includes an internal chamber. In alternate embodiments, the permanent component may comprise a single housing that includes an internal chamber. In an embodiment, the disposable component may comprise a single chamber of varying cross-sectional areas. A thermal transfer pad on the disposable component may comprise a thermal adhesive, pad, membrane, paste, or other material purposed to aid the thermal transfer between the permanent and disposable components.

FIG. 1 is a block diagram illustrating heat exchange system 100 according to an embodiment of the present disclosure. In the example embodiment, heat exchange system 100 is coupled to a thermal management system 110 and operates to regulate the temperature of blood entering via a blood inlet and exiting via a blood outlet. The heat exchange system 100 comprises permanent component 120 and disposable component 150. Disposable component 150 includes thermally conductive membrane 151 adhered on a first surface of disposable component 150 and thermally conductive membrane 152 adhered on a second surface of disposable component 150 opposite the first surface.

Permanent component 120 comprises a first housing 130 that includes a first internal chamber (i.e., Chamber 1) and a second housing 140 that includes a second internal chamber (i.e., Chamber 2). Chambers 1 and 2 receive a thermal fluid (e.g., water or a glycol-based fluid) from the thermal management system 110. The thermal fluid enters housings 130 and 140 from the thermal management system 110 via tubing 131 and 141 and returns to the thermal management system 110 via tubing 132 and 142. In an example embodiment, the thermal management system 110 comprises a heater-cooler device that heats or cools fluid that flows through housing 130 and housing 140, depending on whether the blood flowing through disposable component 150 is being heated or cooled.

As indicated by dotted line arrow 160, disposable component 150 may be inserted into permanent component 120 between housing 130 and housing 140. Once inserted, housing 130 and housing 140 may be opened or closed around disposable component 150, as indicated by the dotted line arrow 170. When closed, a lower surface (not shown) of housing 130 is pressed into contact with thermally conductive membrane 151 of disposable component 150, thereby increasing heat transfer between housing 130 and disposable component 150. Similarly, an upper surface (not shown) of housing 140 is pressed into contact with thermally conductive membrane 152 of disposable component 150, thereby increasing heat transfer between housing 140 and disposable component 150.

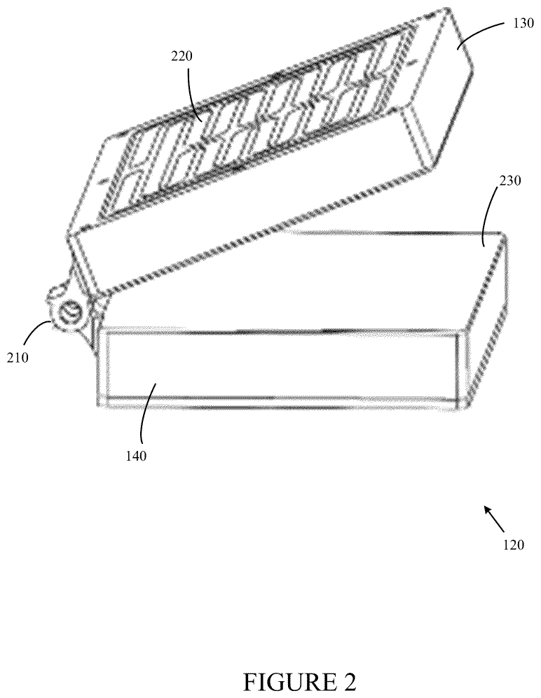

FIG. 2 is a perspective view of a side of permanent component 120 of the heat exchange system 100 according to an embodiment of the present disclosure. In the example embodiment, housings 130 and 140 are substantially block-shaped and are flexibly coupled to each other by a hinge 210. A thermal fluid (e.g., water or a glycol-based solution) is circulated through housing 130 and housing 140 by means of channels within each housing. By way of example, channels 220 within housing 130 may circulate fluid through Chamber 1 in housing 130. Similar channels (not shown) circulate fluid through Chamber 2 in housing 140. FIG. 2 also illustrates the upper surface 230 of housing 140. Surface 230 is pressed into contact with thermally conductive membrane 152 of disposable component 150 when disposable component 150 is enclosed between housing 130 and housing 140.

Housing 130 and housing 140 move between an open position and a closed position by rotating around hinge 210. In an embodiment, hinge 210 is a conventional knuckle joint. More generally, housing 130 and housing 140 may be rotatably coupled to each other by any conventional attachment device, including a plastic strip, a rubber strip, a cable, and the like.

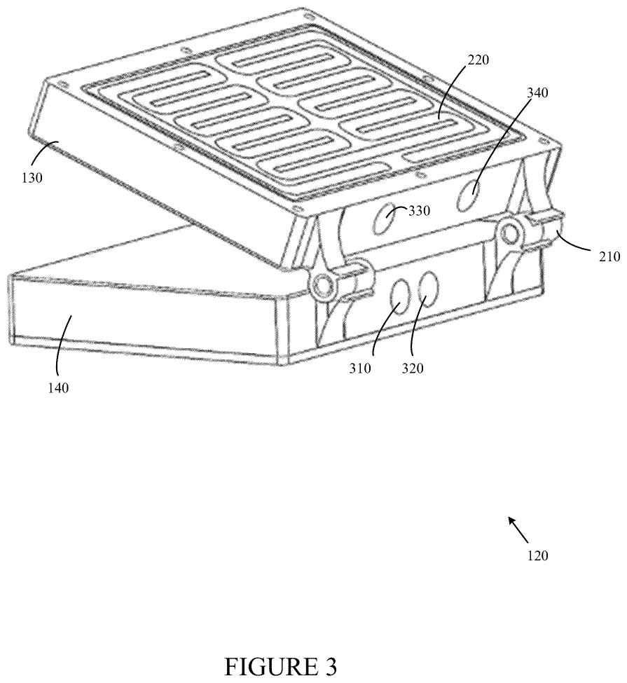

FIG. 3 is a perspective view of the rear of permanent component 120 of heat exchange system 100 according to an embodiment of the present disclosure. In FIG. 3, ports 310 and 330 may be coupled to tubing 131 and 141, respectively, to receive thermal fluid (e.g., water or a glycol-based solution) entering housings 130 and 140, and ports 320 and 340 may be coupled to tubing 132 and 142, respectively, to transfer fluid exiting housings 130 and 140 back to the thermal management system 110. The thermal fluid passing through housings 130 and 140 may increase in temperature if heat is transferred from the blood in disposable component 150 (cooling blood) to the housings 130 and 140 or may decrease in temperature if heat is transferred to blood in disposable component 150 (heating blood) from the housings 130 and 140.

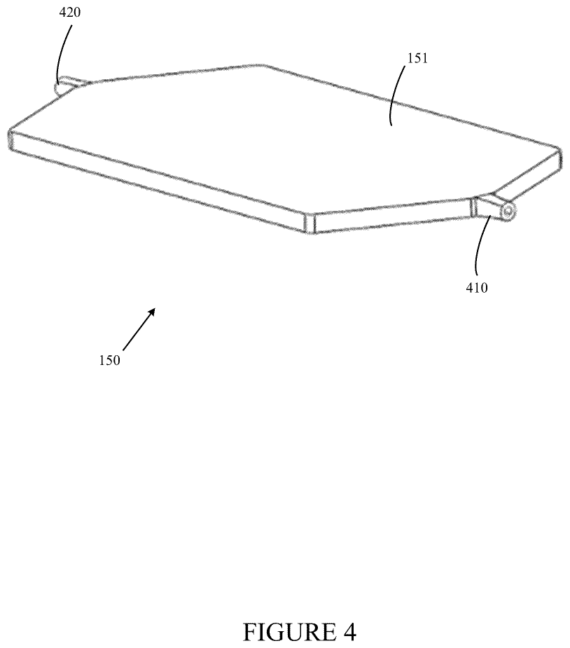

FIG. 4 is a perspective view of disposable component 150 of the heat exchange system 100 according to an embodiment of the present disclosure. In the example embodiment, disposable component 150 comprises a thin, hexagonal chamber that increases the surface area of disposable component 150 so that flowing blood experiences better thermal transfer through the thermally conductive membranes 151 and 152 (not shown). Disposable component 150 comprises inlet port 410 that receives incoming blood and outlet port 420 that outputs outgoing blood. Due to the thinness of disposable component 150, the blood in disposable component 150 is close to both thermally conductive membrane 151 and thermally conductive membrane 152 (not shown), thereby increasing heat transfer. In an exemplary embodiment, the separation between the thermally conductive membrane 151 and the thermally conductive membrane 152 may be less than a quarter inch.

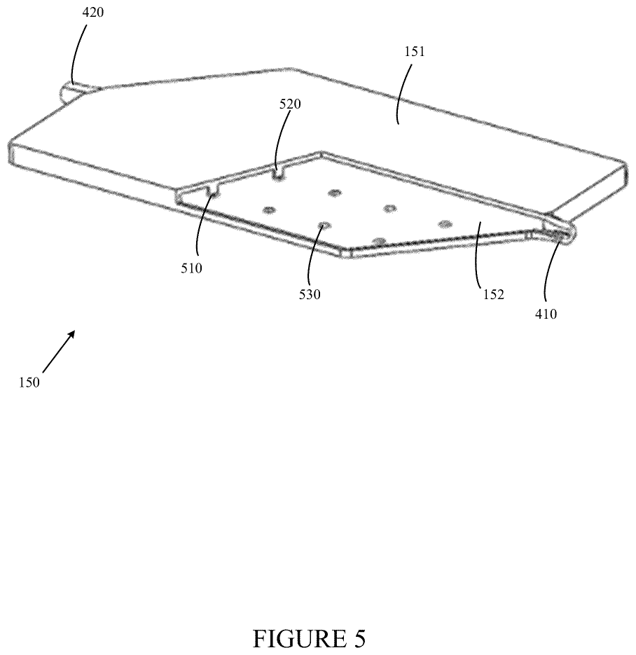

FIG. 5 is a cutaway view of disposable component 150 of the heat exchange system 100 according to an embodiment of the present disclosure. Inside the chamber of disposable component 150, a plurality of posts (or pins), such as example posts 510 and 520, extend between the thermal transfer surfaces of the disposable component 150. Posts 510 and 520 may be mounted in a plurality of dimples (or holes), such as dimple 530. Posts 510 and 520 also assist in transferring heat between the blood inside the chamber and thermally conductive membranes 151 and 152. Further, posts 510 and 520 may provide structural support to the hollowed structure of the disposable component 150.

In FIGS. 1-5 above, permanent component 120 of heat exchange system 100 comprises two substantially block-shaped housings that enclose disposable component 150. Also, disposable component 150 comprises a substantially flat, hexagonal container that maximizes the surface area of disposable component 150 relative to the volume of the internal chamber of disposable component 150. However, this is by way of illustration only and should not be construed to limit the components described herein to a particular geometry. In alternate embodiments, for example, disposable component 150 may be shaped like a circle or an oval, or may be 3-sided (triangle), 4-sided (rectangle), or another number of sides.

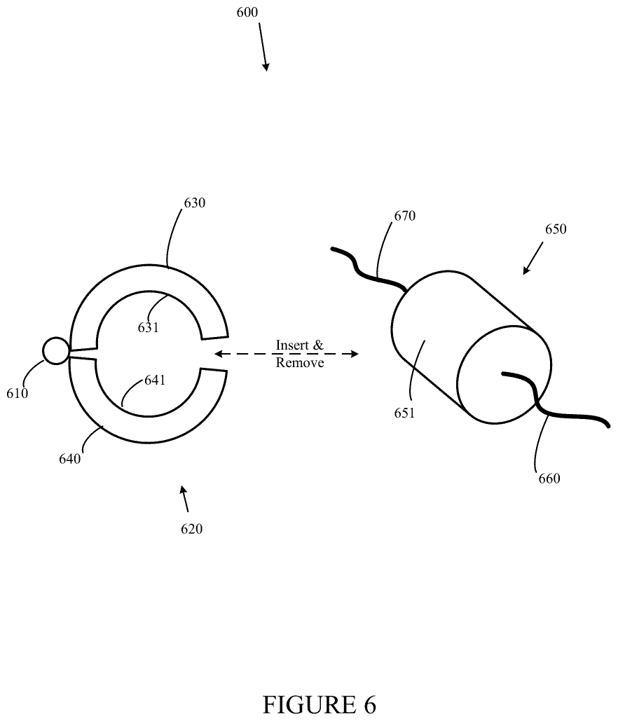

FIG. 6 is a block diagram illustrating heat exchange system 600 according to an alternate embodiment of the present disclosure. In FIG. 6, heat exchange system 600 comprises permanent component 620, which has a hollow cylindrical shape, and disposable component 650, which has a cylindrical shape. Permanent component 620 comprises first housing 630 and second housing 640, both of which have internal chambers that receive a thermal fluid (e.g., water or a glycol-based fluid) from a thermal management system 110. Housings 630 and 640 move between an open position and a closed position by rotating around hinge 610.

As indicated by the dotted line arrow, disposable component 650 may be inserted into permanent component 620 between housing 630 and housing 640. Once inserted, housing 630 and housing 640 may be opened and closed around disposable component 650. When closed, inner surfaces 631 and 641 of housings 630 and 640 are pressed into contact with thermally conductive membrane 651 of disposable component 650, thereby increasing heat transfer between housings 630 and 640 and disposable component 650. Blood inlet 660 and blood outlet 670 circulate blood (or other fluid) through an internal chamber of disposable component 650.

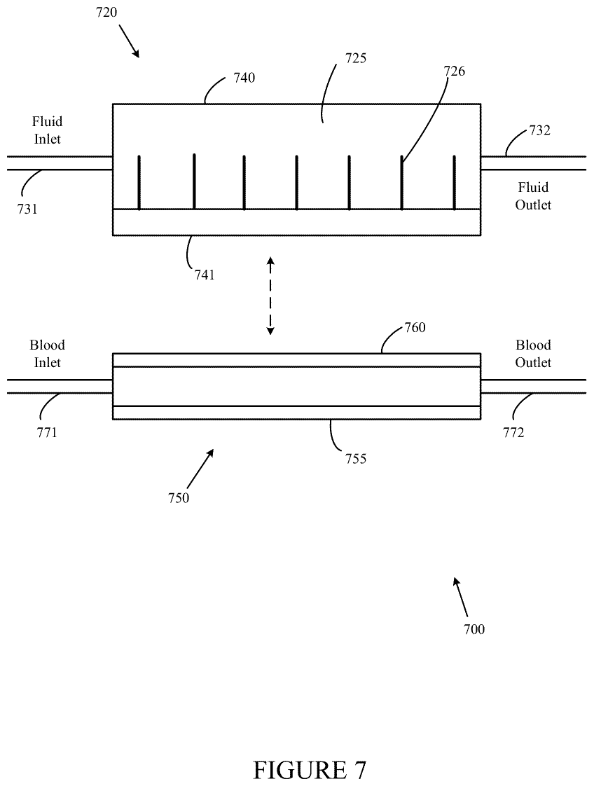

FIG. 7 is a block diagram illustrating heat exchange system 700 according to an alternate embodiment of the present disclosure. The heat exchange system 700 comprises permanent component 720 and disposable component 750. Permanent component 720 and disposable component 750 may both be rectangularly shaped blocks, as in FIG. 1. However, in FIG. 7, permanent component 720 comprises fluid inlet 731 and fluid outlet 732 and only a single housing 740. Housing 740 encloses internal chamber 725 and comprises a plurality of thermally conductive fins, such as fin 726, that improve heat transfer between the thermal fluid in chamber 725 and the thermally conductive lower surface 741 of housing 740.

Disposable component 750 comprises thermally conductive surface 760 and insulation layer 755. Blood inlet 771 and blood outlet 772 circulate blood (or other fluid) through an internal chamber of disposable component 750. As indicated by the dotted line arrow, disposable component 750 may be pressed into contact with permanent component 720 such that thermal transfer may occur between thermally conductive surface 741 of housing 740 and thermally conductive surface 760 of disposable component 750.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A heat exchange system configured to control the temperature of blood of a patient, the heat exchange system comprising:
a disposable component comprising a blood inlet configured to receive blood from the patient and a blood outlet configured to return blood to the patient; and
a permanent component including:
a first housing comprising an inlet and an outlet configured to exchange thermal fluid with an external device; and
a second housing comprising an inlet and an outlet configured to exchange thermal fluid with the external device,
wherein the disposable component is configured to be inserted between the first housing and the second housing, such that in a closed position of the permanent component, a first surface of the disposable component is pressed into contact with a surface of the first housing and a second surface of the disposable component is pressed into contact with a surface of the second housing.

2. The heat exchange system as set forth in claim 1, wherein the surface of the first housing comprises a thermally conductive membrane.

3. The heat exchange system as set forth in claim 2, wherein the surface of the second housing comprises a thermally conductive membrane.

4. The heat exchange system as set forth in claim 3, wherein the first and second surfaces of the disposable component comprise thermally conductive membranes.

5. The heat exchange system as set forth in claim 4, wherein the first and second surfaces of the disposable component are separated by less than a quarter inch.

6. The heat exchange system as set forth in claim 5, wherein the first and second surfaces of the disposable component are substantially flat.

7. The heat exchange system as set forth in claim 1, wherein the permanent component comprises an attachment device for coupling the first housing and the second housing.

8. The heat exchange system as set forth in claim 7, wherein the attachment device comprises a hinge that rotatably couples the first and second housing.

9. The heat exchange system as set forth in claim 8, wherein the hinge is configured to rotate the first housing and the second housing between the closed position and an open position.

10. The heat exchange system as set forth in claim 1, wherein the first housing and the second housing are configured to form a hollow cylinder and the disposable component comprises a cylinder.
